(19) United States
(12) Patent Application Publication
(10) Pub. No.: US 2026/0053564 A1
(43) Pub. Date: Feb. 26, 2026

(54) SMALL OPTICAL CORE HYBRID FIBER FOR SURGICAL LASER PROCEDURES SUCH AS LASER LITHOTRIPSY THAT UTILIZE HOLMIUM YAG LASERS AND/OR THULIUM FIBER LASERS

(71) Applicant: OPTICAL INTEGRITY, INC., Panama City Beach, FL (US)

(72) Inventors: Joe D. BROWN, Panama City Beach, FL (US); Daniel MALPHURS, Panama City Beach, FL (US); Howard S. KLYMAS, Panama City Beach, FL (US)

(21) Appl. No.: 19/306,588

(22) Filed: Aug. 21, 2025

Related U.S. Application Data
(60) Provisional application No. 63/685,404, filed on Aug. 21, 2024.

(51) Int. Cl. A61B 18/26; A61B 18/22

(52) U.S. Cl. CPC A61B 18/26; A61B 2018/2205; A61B 2018/2244; A61B 2018/2294; A61B 2218/002

(57) ABSTRACT

A surgical laser fiber for use in surgical laser procedures such as laser lithotripsy includes a relatively small diameter silica core surrounded by a thin intermediate doped silica cladding and a relatively thick outer glass cladding or ferrule surrounding the thin intermediate doped silica cladding, with the result that erosion of the fiber is primarily confined to the silica core, causing the relatively thick outer glass cladding or ferrule to form a standoff that extends beyond the eroded end of the silica core as lasing proceeds. The diameter of the silica core may be approximately 80 μm and a thickness of the outer glass cladding may be approximately 200 μm. The surgical laser fiber may be used with Thulium Fiber Lasers, or may be adapted for use with both Thulium Fiber Lasers and Holmium YAG lasers.

### BACKGROUND OF THE INVENTION

This application claims the benefit of U.S. Provisional Patent Application Ser. No. 63/685,404, filed Aug. 21, 2024, and incorporated by reference herein.

### 1. FIELD OF THE INVENTION

The invention relates to the field of laser therapy, treatment, or surgery, and in particular to optical fiber arrangements adapted to mitigate the effects of fiber erosion occurring during surgical laser procedures.

The invention may be used in connection with high frequency lasers such as Thulium Fiber Lasers (TFLs), lower frequency lasers such as Holmium YAG lasers, or combo systems that include both low and high frequency lasers.

The surgical laser fibers and method of the invention is especially suitable for use in laser lithotripsy procedures but may also be used, or have features applicable to, surgical procedures other than lithotripsy, including procedures involving lasing of objects or tissues other than urological stones.

### 1. DESCRIPTION OF RELATED ART

As described by way of example in commonly owned U.S. Pat. No. 11,109,911 (“Stone Sense with Fiber Erosion Protection and Camera Saturation Prevention, and/or Absence-Detection Safety Interlock”); U.S. Pat. No. 11,172,988 (“End Fire Fiber Arrangements With Improved Erosion Resistance”); U.S. Pat. No. 11,278,352 (“Protective Caps or Tips for Surgical Laser Fibers”); and U.S. Pat. No. 11,376,071 (“Method of Reducing Retro-Repulsion During Laser Lithotripsy”) overheating and damage to a fiber tip can result from a phenomenon known as free election absorption (FEA), which occurs when the temperature at the laser fiber tip exceeds approximately 1000° C. In Holmium YAG laser lithotripsy systems, which have a relatively low frequency of 100 Hz or less, FEA-inducing increases in temperature typically occur when the tip of the fiber is adjacent to, or comes into contact, with the stone. To prevent such contact, U.S. Pat. No. 11,278,352 proposed to place a standoff sleeve at the end of fiber, in order to maintain a predetermined minimum spacing between the fiber tip and the stone being targeted. A commercial version of the standoff described in U.S. Pat. No. 11,278,352 is currently sold by Optical Integrity, Inc. under the tradename Excalibur™.

Fibers with the Excalibur™ standoff have a core diameter of 200 to 272 μm or microns. FIG. 1A shows such a fiber, including the relatively large silica core 10 and a relatively thin doped silica cladding layer 12, while FIG. 1B illustrates the effect of FEA when the tip of the fiber contacts a kidney stone 15 targeted for destruction. When the fiber end surface or tip 13 contacts the kidney stone 15, FEA causes the large silica core 10 and doped silica cladding layer 12 of the laser fiber to self-absorb all of the laser energy, raising the temperature of the fiber tip to approximately 5000° C., producing a bright visible light and quickly eroding the distal fiber tip surface 13 shown in FIG. 1A, and resulting in the jagged surface 13′ shown in FIG. 1B.

FIG. 1C depicts a standoff sleeve 50 in the form of an ETFE capillary of the type described in U.S. Pat. No. 11,278,352, which is positioned at the end of the fiber to maintain a minimum spacing between the stone and the fiber. The silica fiber tip 51 is recessed within the ETFE capillary, with the resulting separation between fiber tip and stone helping to keep the average temperature down and limit erosion of the fiber tip. The soft ETFE material of the standoff sleeve 50 also protects the endoscope's fragile working channels.

While the soft standoff sleeve is effective to reduce erosion in Holmium YAG laser applications, however, conventional Holmium YAG lasers are currently being replaced by Thulium Fiber Lasers (TFLs}, which have a much higher frequency than Holmium YAG lasers (more than 10 times higher). Use of a higher frequency laser in lithotripsy procedures has a number of advantages, including quicker and more complete stone destruction. More rapid stone destruction shortens lithotripsy procedure times, while the smaller spot size of TFLs results in the creation of smaller stone fragments or particles, which are more easily excreted by the patient.

However, the inventors have found that the higher power of TFLs can result in surgical site temperatures high enough to cause FEA and erode the fiber tip even in the absence of contact between the fiber tip and the laser. Furthermore, the higher temperatures can actually cause a standoff made of a material such as ETFE to melt, with catastrophic results.

Thus, while an advantage of higher frequency TFL lasers is that they can produce finer particles or dust during lithotripsy (and in theory allow use of smaller core fibers that direct the energy more precisely), lithotripsy practitioners or surgeons have found that, in practice, the smaller the core fiber, the faster the erosion. Initially, it was though that the switch to TFLs would enable use of a smaller 150-micron core fiber to achieve higher energy density and more efficient dusting, but surgeons soon found that the smaller core fiber eroded too fast, and as a result lithotripsy surgeons moved back to 200 or 272-micron cores. Consequently, erosion due to high heat remains a problem for surgeons using TFLs. To make matters worse, the FDA has mandated recalls for manufacturers to pull fiber strippers because they cannot be validated for reuse in the sterile field. In the absence of validation for re-use, doctors would have to dispose of the strippers after a single use, rendering attempts to deal with fiber erosion by stripping and cleaving the fiber during a lithotripsy procedure economical impractical due to the high cost of the strippers.

In the case of Holmium lasers with a peak power of 15 kw, Dr. Traxer has proposed to use scissors to simply cut the eroded end of a 200-273 micron core fiber to re-terminate the fiber with no stripping or cleaving. While the end face of the cut fiber is not as good as a cleaved face, it still has enough power density to destroy stones. However, a thulium fiber laser has a peak power of only 500 watts with a 200-273 micron core fiber, and therefore the power density can be too low to destroy stones with a scissor cut, precluding the use of this low cost method of re-terminating fibers when using thulium fiber lasers.

As illustrated in FIG. 2A, one solution to the problems resulting from the higher temperatures that occur when using TFLs in place of Holmium YAG lasers is to replace the ETFE soft tip 50 of FIG. 1C with a high temperature resistant silica capillary standoff 14, as shown in FIG. 2A, to maintain spacing between the stone and fiber. Such a high temperature resistant standoff is described in commonly owned U.S. Patent Publication No. 2019/0038355, entitled “End Fire Fiber Arrangements with Improved Erosion Resistance,”and sold by Optical Integrity under the tradename Excalibur™ TFL.

In the arrangement of FIG. 2A, the pure silica capillary 14 is welded to the thin doped silica cladding 12 that surrounds the pure silica core 10. However, while the arrangement of FIG. 2 reduces erosion, the dust trapped inside the capillary can still produce FEA erosion of the fiber core face 10 and the capillary's inside diameter 14. The erosion creates an undesired reduced power density.

The inventors have addressed the problem of trapped dust in a variety of ways, including the use of reflective standoffs, standoffs with openings for the removal of dust, and the use of low power cleaning laser pulses or continuous waves, and/or fluid, to expel the dust. These solutions are described, for example, in commonly owned U.S. Patent Publication Nos. 2023/0101488 (“Surgical Laser Fiber with Reflective Standoff Sleeve and Method of Preventing Dust Particle Buildup with a Standoff Sleeve”); 2024/0164836 (“Surgical Laser Fiber Standoff Arrangement for Preventing Dust Particle Accumulation During a Laser Lithotripsy Procedure”); and 2019/0201100 (“2019/0201100 (“Method of Reducing Retro-Repulsion During Laser Lithotripsy”); and U.S. Pat. No. 11,253,318 (“Arrangement for Filtering Out Damaging Heat Created from Laser Energy Contacting a Kidney Stone”).

The present invention provides another, simpler way to mitigate the effects of high temperatures and FEA during laser lithotripsy procedures, whether using Holmium YAG lasers or TFLs. The invention can be used alone or in connection with one or more of the above-described arrangements.

### SUMMARY OF THE INVENTION

It is accordingly a first objective of the invention to provide surgical laser fiber arrangements that reduce FEA-induced fiber erosion, and/or that mitigate the effects of fiber erosion, during surgical laser procedures such as, by way of example and not limitation, laser lithotripsy.

It is a second objective of the invention to provide surgical laser fiber arrangements that enable cleaving of fibers during a procedure without the need for stripping of the fiber.

It is a third objective of the invention to provide laser lithotripsy systems that can be used with high frequency Thulium Fiber Lasers (TFLs) as well as lower frequency Holmium YAG lasers.

It is a fourth objective of the invention to provide a laser fiber arrangement suitable for use with high frequency laser systems that enables use of small core diameter laser fibers without requiring modification of the laser system itself.

It is a fifth objective of the invention to provide a simple way to reduce fiber erosion that can be used alone or in combination with other fiber erosion reduction arrangements such as silica standoffs, low frequency cleaning pulses, modified standoffs with openings for the expulsion of dust, and so forth, including the arrangements described in the above-cited patents and publications.

It is a sixth objective of the invention to enable re-termination of TFL fibers during a lithotripsy procedure without the need for stripping and cleaving of the fibers, by cutting the fibers using scissors, while still providing sufficient power density to destroy stones and limit erosion rates.

These objectives are achieved, in accordance with the principles of an exemplary embodiment of the invention, by a novel laser lithotripsy fiber having an increased cladding-to-core diameter ratio, with a smaller core that produces smaller stone fragments and at the same time self-creates a silica capillary standoff, slowing the erosion rate. As with conventional lithotripsy fibers, when the fiber erodes sufficiently to cause a significant reduction in power density, the fiber may be cleaved, allowing re-use of the fiber, but the increased core-to-cladding ratio can reduce the need for cleaving.

By way of example and not limitation, a surgical laser fiber constructed according to the principles of the invention may have a core diameter of approximately 80 μm and a cladding thickness of 200 μm surrounding a small intermediate doped cladding layer.

According to a variation of the exemplary embodiment, the extra-large cladding may be replaced by an equally thick silica ferrule welded to the cladding layer. While adhering a ferrule or any other structure to the end of the fiber eliminates the possibility of cleaving the fiber, erosion might be sufficiently reduced that cleaving is unnecessary.

According to another variation of the exemplary embodiment, in applications where the power density is too low or erosion is so fast that melting of the fiber buffer is possible, the end of the fiber may be pre-stripped and the fiber enclosed in a sheath. Suitable sheaths are disclosed in, but not limited to, the fiber sheath described in commonly owned U.S. Patent Publication Nos. 2013/0218147; 2014/0316397; and 2015/0148789. When melting occurs, the stripped end of the fiber may be extended from the sheath and cleaved by a cleaver that has been pre-sterilized for use in the sterile environment and that may be discarded after use. Furthermore, despite a coarser cut, the present invention allows the use of scissors rather than more-expensive cleavers to re-terminate TFL fibers because of the higher power density output of the small core, large cladding fiber.

In yet another variation, the increased cladding-to-core ratio surgical laser fiber of the exemplary embodiment may further include a silica capillary and silica standoff structure that helps limit erosion and provides a hollow waveguide or “Moses” configuration of the type disclosed in, for example, commonly owned U.S. Pat. No. 11,376,071, cited above.

In still further variations of the exemplary embodiment, fibers with increased core-to-cladding ratio may be combined with protective ferrules or capillary structures having reflectors or diffusers to ensure that only high power density energy gets launched into the smaller core and only high energy density power is emitted from the distal end of the fiber.

Variations of the reduced core diameter fiber arrangements of the present invention may be used in connection with high frequency lasers such as Thulium Fiber Lasers (TFLs), lower frequency lasers such as Holmium YAG lasers, or combo systems that include both low and high frequency lasers. In addition, the fiber arrangements and methods described herein may have applicable to surgical lasers other than Holmium or TFL laser, surgical procedures other than laser lithotripsy, and lasing of objects or tissues other than urological or kidney stones.

### BRIEF DESCRIPTION OF THE DRAWINGS

FIG. 1A is a cross-sectional side view of a conventional surgical laser fiber used in laser lithotripsy procedures.

FIG. 1B is a cross-sectional side view showing the effects of FEA erosion on the surgical laser fiber of FIG. 1A.

FIG. 1C depicts a conventional solution to the FEA erosion illustrated in FIG. 1B, in which an ETFE standoff is used to maintain spacing between the tip of the fiber and a stone.

FIG. 2A shows a variation of the conventional solution shown in FIG. 1C, in which the soft tip is replaced by a silica standoff.

FIG. 2B is a cross-sectional side view of a surgical laser fiber with a silica standoff, in which the conventional fiber is replaced by a small core, large cladding, high power density fiber according to the principles of an exemplary embodiment of the present invention.

FIG. 2C is a cross-sectional side view of a variation of the small core surgical laser fiber construction of FIG. 2C, with a large secondary cladding.

FIGS. 3A and 3B are cross-sectional side views showing another example of a surgical laser fiber with an enlarged cladding-to-core diameter ratio constructed of the type shown in FIGS. 2B and 2C.

FIGS. 3C and 3C2 show the effects of FEA erosion on the surgical laser fiber of FIGS. 3A and 3B.

FIG. 3C3 is a cross-sectional side view of the surgical laser fiber of FIGS. 3A to 3C2 combined with a silica standoff.

FIG. 3D is a cross-sectional side view of a variation of the surgical laser fiber of FIG. 3A to 3C2, positioned within a sheath.

FIG. 3E shows the surgical laser fiber of FIG. 3E in an extended position.

FIG. 3F shows the effects of FEA erosion on the extended surgical laser fiber of FIG. 3E.

FIG. 3G shows the eroded surgical laser fiber of FIG. 3F, after cleaving or cutting.

FIG. 4A is a cross-sectional side view showing a variation of the surgical laser fiber of FIG. 2C with the addition of a silica standoff and a fluid port.

FIG. 4B shows an extended waveguide version of the silica standoff of FIG. 4A.

FIG. 4C is a cross-sectional side view showing a variation of the surgical laser fiber of FIG. 2C with a metal standoff having a port.

FIG. 4D shows the surgical laser fiber of FIG. 4C with the addition of a sheath.

FIG. 5A illustrates the output of the surgical laser fiber of FIG. 3A.

FIG. 5B is a cross-sectional side view of a hybrid version of the surgical laser fiber of FIG. 5A, which can be used with both high frequency lasers such as TFLs, and lower frequency lasers such as Holmium YAG lasers.

FIG. 6 is a cross-sectional side view of a variation of the exemplary embodiment with added high pass filter in the form of a reflector or diffuser.

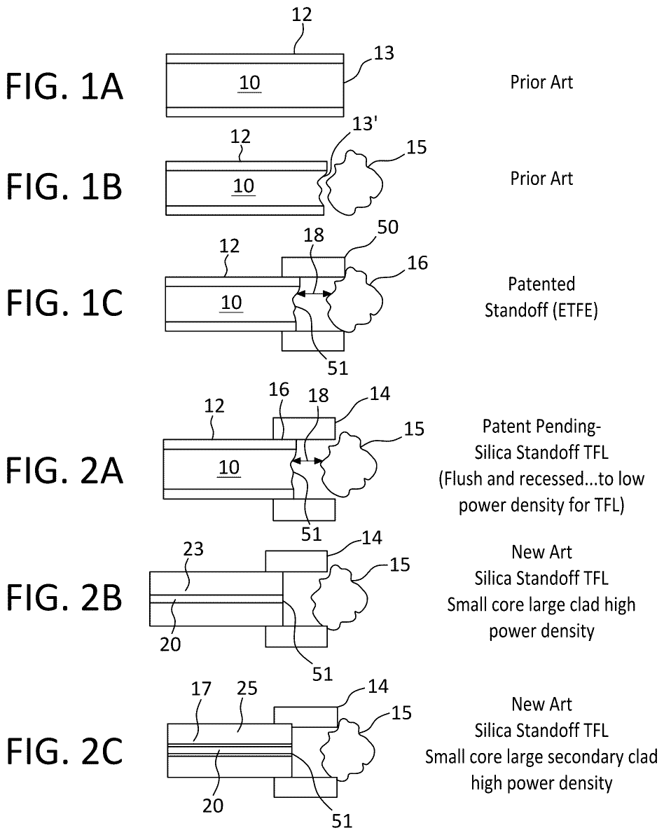

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

According to an exemplary embodiment of the invention shown in FIGS. 2B and 2C, the conventional surgical laser fiber with a 200 to 270 μm core is replaced by a surgical laser fiber with a much smaller 80 micron core 20. Consequently, the illustrated fibers produce a high-power density when used for laser lithotripsy, which results in smaller stone fragments with a lower energy laser. In the example illustrated in FIG. 2B, the conventional thin doped silica cladding 12 of FIG. 2A is replaced by an extra-large glass cladding 23 that surrounds the relatively small core and that has a thickness of 200 μm. In the variation shown in FIG. 2C, the single cladding layer 23 is replaced by an extra-large outer glass cladding 25 and a small or thin intermediate doped cladding layer 17 surrounding the 80 micron core 20. In both examples, distance to the stone 15 is maintained by a silica standoff capable of withstanding the high temperatures that occur when the fiber is used with a Thulium Fiber Laser.

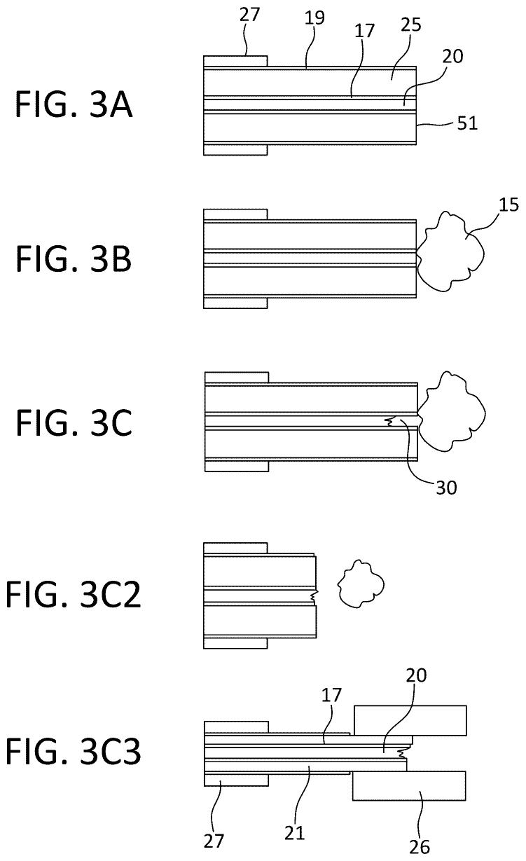

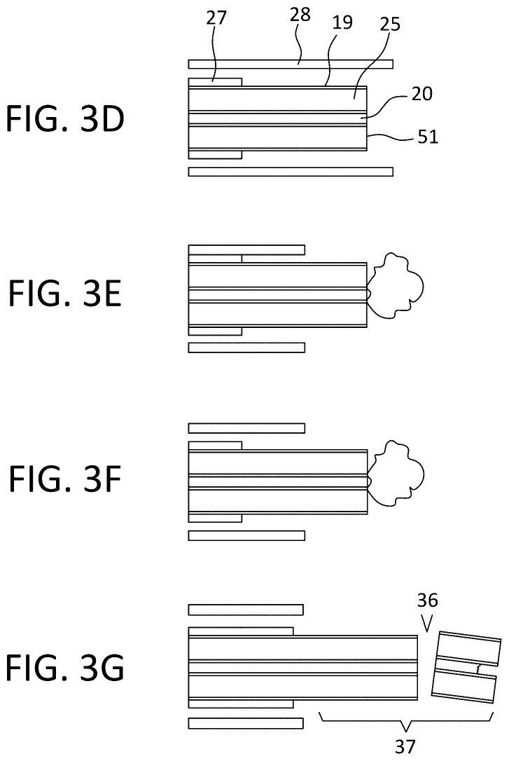

FIGS. 3A to 3C illustrate the use of the small core, large clad fiber of FIGS. 2B and 2C without an added silica standoff, but which has the effect of self-creating the equivalent of a standoff. The fiber of this example includes an 80 micron core 20, an intermediate doped cladding layer 17, a large outer glass cladding 25 having a thickness of at least 200 μm, a coating 19, and a buffer 27 that has been stripped back from the tip 51 of the fiber. Initially, the stone 15 may come into contact with the tip or end face 51 of the fiber, as shown in FIG. 3B. Since erosion occurs primarily in the core, continued lasing will cause the end of the core to erode back from end of the silica cladding, slowing the erosion rate as lasing continues.

As a result, in this example, instead of requiring a separate standoff, continued lasing of the stone 15 has the effect of self-creating a silica capillary standoff 30 consisting of a portion of the cladding 25 within which the core 20 has eroded away. Furthermore, when continued lasing against the stone 15 eventually erodes the entire fiber tip as shown in FIG. 3C2, the erosion process repeats to maintain the self-created standoff 30, in effect “resetting” the fiber. The relatively small core 20 (in comparison with conventional fiber cores) keeps the power density high enough to maintains a destruction threshold until the fiber “resets,” even as the increased power density allows the physician operating the laser to achieve stone destruction from a further distance between the fiber tip and the stone, resulting in still further slowing of the erosion rate.

In a variation of the example shown in FIGS. 3A to 3C and 3C2, the extra-large or thick cladding 25 of FIGS. 3A and 3B may be replaced by an extra-large silica ferrule 26 welded or otherwise adhered to a relatively thin cladding layer 21 surrounding the 80 μm core, as shown in FIG. 3C3. This variation allows the same high-power density as the example shown in FIGS. 3A and 3B, but because the ferrule 26 is limited to the tip of the fiber, rather than extending the entire length of the fiber as does the cladding 25 of FIGS. 3A and 3B, the arrangement of FIG. 3C3 eliminates the possibility of re-cleaving the fiber. As a result, when using this variation, additional measures may be necessary to remove build-up of debris within the ferrule, such as (by way of example and not limitation) the dust-removal or build-up prevention measures described in commonly owned U.S. Patent Publication Nos. 2023/0101488; 2024/0164836; and 2019/0201100; and U.S. Pat. No. 11,253,318, cited above and incorporated herein by reference.

In either example, the 80 μm core 20 produces a much smaller particle size than the larger 200 to 270 μm core of the conventional fiber, allowing not only lower joules but also increased frequency, resulting in much better stone dusting efficiency. In addition, the increased power density from a smaller core helps destroy hard stones and minimizes carbon formation in kidney stones when using relatively low power Thulium fiber lasers, which have power peaks of around 500 Watts using a 272 μm core fiber, compared to a Holmium YAG laser, which has a peak of around 15 kw for the same fiber core diameter. This helps solve the problem that stone destruction can be impeded by black carbon spots formed from organics on the stone surface when the power density is below the stone destruction threshold, which is more of a problem with Thulium lasers than lower frequency, higher power Holmium lasers. Once the spots are formed continued laser pulses only dry, rather than destroy, the stone, so it is important to prevent black carbon spot formation in the first place.

As shown in FIGS. 3D to 3G, if continued erosion of the small core fiber 20 with an extra-large cladding 25 still causes a power density that is too low or erosion that is too fast, to the point where the fiber buffer 27 begins to melt, a sheath 28 of the type described in the above-cited commonly owned U.S. Patent Publication Nos. 2013/0218147; 2014/0316397; and 2015/0148789, or any other corresponding sleeve or sheath, may be added. This will help protect both the fiber itself and the scope through which the fiber is inserted during a procedure.

As erosion reduces the power density, the fiber can be re-cleaved as needed by extending the eroded pre-stripped section shown in FIG. 3F from the sheath 28 and cleaving the exposed section 37 at location 36, as shown in FIG. 3G. While stripping of the fiber is not a validated method for use in a sterile environment, cleavers are inexpensive and can be provided in validated and sterile form for use with each single-use disposable fiber, eliminating the need to replace the entire fiber during a stone-removal procedure.

Furthermore, in this example, it may even be possible to eliminate the need for a cleaver, and instead use inexpensive scissors to re-terminate fibers during a lithotripsy procedure using TFL fibers. While the irregular nature of a scissors cut would preclude use in connection with TFL fibers because of their low power density in comparison with Holmium laser fibers, the small core, large cladding fibers of the exemplary embodiment provide a sufficiently initial power density that the exemplary fibers can still be used to destroy stones despite reductions in power density when re-terminated by scissors.

Whether the “standoffs” illustrated in FIGS. 3C and 3F are added-on self-created, a continuous air space or bubble can be created in the standoff or ferrule that reduces attenuation of the output laser beam in a manner similar to the “Moses” effect described in commonly owned U.S. Pat. No. 11,376,071.

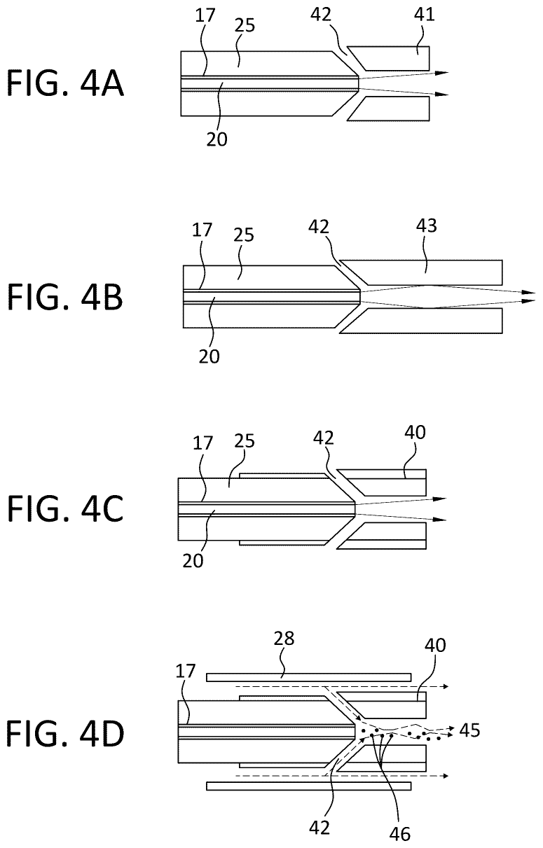

In a further variation of the exemplary embodiment of the invention, the silica standoff 14 of FIG. 2C may be replaced by a combination of a silica capillary 41, shown in FIGS. 4A and 4B or a metal standoff 40 shown in FIGS. 4C and 4D, that has been welded or otherwise adhered to the fiber cladding (or buffer). To allow fluid flow, the silica standoff 41 or metal standoff 40, may be provided with a port 42 to allow fluid from a scope (not shown) and/or a sheath 28. For example, the addition of a sheath 28 with water flow 45, as shown in FIG. 4D, can substantially improve the rinsing process of removing debris 46 from the stone destruction procedure. Port 42 may be positioned to maximize laser transmission.

In the configuration shown in FIG. 4B, the standoff 41 has been extended and acts as a hollow waveguide 43. The metal standoff shown in FIGS. 4C and 4D could similarly be extended to serve as a waveguide when made of a reflective metal or coated with reflective materials such as aluminum, gold, silver, or the like.

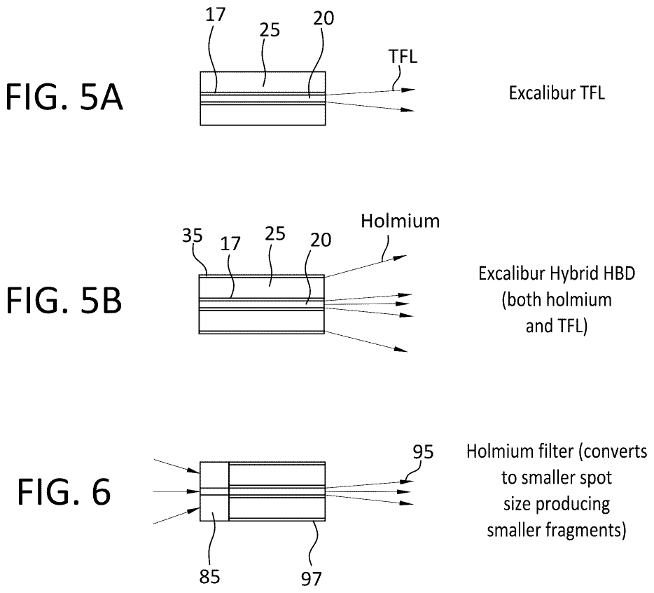

FIG. 5A shows a surgical laser fiber with an enlarged cladding thickness corresponding to that of the exemplary fiber shown in FIG. 3A which, when used with a TFL laser, provides the high density output indicated by arrows labeled “TFL. ” This configuration can be modified, as shown in FIG. 5B, by adding an extra doped cladding layer 35 that acts as a secondary waveguide, allowing the fiber to be used with hybrid combo lasers such as a Holmium: Yag/Thulium Fiber lasers, with the Holmium laser output indicated by the arrows labeled “Holmium.” With one fiber, the lithotripsy surgeon can launch both individually or simultaneously a Holmium laser with a larger spot size (e.g., 200 μm) and a larger numerical aperture (>0.2), as well as a Thulium fiber laser with a spot size of 50 microns and numerical aperture of less than 0.2. The 80 μm core helps maintain high power density for both wavelengths, while the smaller core size creates smaller particle sizes for any given wavelength.

With the hybrid fiber and a hybrid laser system containing a Holmium and a Thulium laser, both wavelengths could be used to create a continuous air space, also known as a “Moses” effect, in a standoff ferrule such as, by way of example and not limitation, ferrule 14 of FIGS. 2B and 2C or waveguide 43 of FIG. 4B.

In another variation of the exemplary embodiment of the invention, the small core/large cladding fiber may be combined with a diffuser and/or reflector 85 to filter focused radiation, as shown in FIG. 6, and ensure that only high power density energy 95 is launched into the small core 20, and that only high energy density power is emitted from the distal end 97. Reflecting or diffusing low-density energy while allowing passage of high-density energy at higher frequencies creates smaller stone fragmentation while helping to keep the temperature of the stone destruction site in the kidney or ureter down, reduce retrorepulsion, and also lower the output numerical aperture to make the waveguides more reflective.

What is claimed is:

1. A surgical laser fiber for use in a surgical laser procedure, comprising:
a relatively small diameter silica core surrounded by a thin intermediate doped silica cladding; and
a relatively thick outer glass cladding surrounding the thin intermediate doped silica cladding,
wherein erosion of the fiber is primarily confined to the silica core, causing the relatively thick outer glass cladding to form a standoff that extends beyond the eroded end of the silica core as lasing proceeds.

2. The surgical laser fiber as claimed in claim 1, wherein a diameter of the silica core is approximately 80 μm and a thickness of the outer glass cladding is approximately 200 μm.

3. The surgical laser fiber as claimed in claim 1, wherein the surgical laser fiber is coupled to a Thulium Fiber Laser (TFL).

4. The surgical laser fiber as claimed in claim 1, wherein the surgical laser procedure is a lithotripsy procedure.

5. The surgical laser fiber as claimed in claim 4, wherein the surgical laser fiber is pre-stripped and movably positioned in a sheath so that the surgical laser fiber can be extended from the sheath for cleaving without re-stripping when output power density drops due to fiber erosion during the lithotripsy procedure.

6. The surgical laser fiber as claimed in claim 1, wherein a silica, metal, or reflectively coated standoff is fixed to the outer glass cladding.

7. The surgical laser fiber as claimed in claim 6, wherein the standoff in configured as a waveguide.

8. The surgical laser fiber as claimed in claim 6, wherein the standoff further includes a fluid irrigation port.

9. The surgical laser fiber as claimed in claim 1, further comprising a second relatively thick doped cladding surrounding the relative thick glass cladding, wherein the second relatively thick doped cladding acts as a secondary waveguide to enable use of the surgical laser fiber with either a TFL or a Holmium:YAG laser.

10. The surgical laser fiber as claimed in claim 9, further comprising a filter element for reflecting or dissipating lower laser power density.

11. The surgical laser fiber as claimed in claim 10, wherein the surgical laser fiber is positioned in a sheath from which the surgical laser fiber may be extended for cleaving during the surgical laser procedure.

12. The surgical laser fiber as claimed in claim 11, wherein a standoff and/or waveguide is fixed to the sheath to allow irrigants to clean and cool a tip of the surgical laser fiber.

13. A surgical laser fiber for use in a surgical laser procedure, comprising:
a relatively small diameter silica core surrounded by a thin intermediate doped silica cladding; and
a relatively thick ferrule adhered to and surrounding the thin intermediate doped silica cladding,
wherein erosion of the fiber is primarily confined to the silica core, causing the relatively thick outer glass cladding to form a standoff that extends beyond the eroded end of the silica core as lasing proceeds.

14. The surgical laser fiber as claimed in claim 13, wherein a diameter of the silica core is approximately 80 μm and a thickness of the outer glass cladding is approximately 200 μm.

15. The surgical laser fiber as claimed in claim 13, wherein the surgical fiber is coupled to a Thulium Fiber Laser (TFL).

16. The surgical laser fiber as claimed in claim 13, further comprising a filter element for reflecting or dissipating lower power density laser.

17. The surgical laser fiber as claimed in claim 13, wherein the surgical laser fiber is movably positioned in a sheath so that the surgical laser fiber can be extended from the sheath for cleaving when output power density drops due to fiber erosion during a lithotripsy procedure.

18. The surgical laser fiber as claimed in claim 13, wherein the ferrule is a glass ferrule that extends beyond an end face of the core and intermediate doped cladding.

19. A laser lithotripsy method, comprising the steps of:
providing a surgical laser fiber having a relatively small diameter silica core and either a relatively thick cladding or a relatively thick ferrule adhered to and surrounding a thin intermediate doped silica cladding;
pre-stripping an end of the surgical laser fiber;
utilizing the surgical laser fiber to destroy a stone during a laser lithotripsy procedure using a thulium and/or holmium laser; and
re-terminating the surgical laser fiber during the procedure to remove an eroded section of the pre-stripped end of the surgical laser fiber.

20. The laser lithotripsy method of claim 18, wherein the laser lithotripsy procedure
uses a thulium laser and the surgical laser fiber is re-terminated by using pre-sterilized scissors to cut the eroded section of the pre-stripped end of the surgical laser fiber.
